(19) United States
(12) Patent Application Publication
(10) Pub. No.: US 2024/0278220 A1
(43) Pub. Date: Aug. 22, 2024

(54) MOLTEN IRON CATALYST FOR PRODUCING HIGH-CARBON ALPHA-OLEFINS FROM SYNTHESIS GAS AND PREPARATION METHOD AND APPLICATION THEREOF

(71) Applicant: Yankuang Energy R&D Co., Ltd., Shanghai (CN)

(72) Inventors: Qiwen Sun, Shanghai (CN); Yan Sun, Shanghai (CN); Zongsen Zhang, Shanghai (CN); Angjun Chen, Shanghai (CN); Qiang Teng, Shanghai (CN); Xiaochun Cao, Shanghai (CN)

(73) Assignee: Yankuang Energy R&D Co., Ltd., Shanghai (CN)

(21) Appl. No.: 18/572,001

(22) PCT Filed: Mar. 2, 2022
(86) PCT No.: PCT/CN2022/078740

§ 371 Date: Dec. 19, 2023

(30) Foreign Application Priority Data
Dec. 14, 2021 (CN) 202111525228.9

(51) Int. Cl. B01J 23/889; B01J 35/27; B01J 37/00; C10G 2/00

(52) U.S. Cl. CPC B01J 23/8892; B01J 35/27; B01J 37/0036; B01J 37/0081; C10G 2/332; C10G 2400/22

(57) ABSTRACT

The present application relates to the technical field of chemical production, and particularly relates to a molten iron catalyst for high-temperature Fischer-Tropsch synthesis, a preparation method of the molten iron catalyst and an application of the molten iron catalyst in preparation of high-carbon α-olefin from synthesis gas. The molten iron catalyst comprises iron oxides and a cocatalyst, and mass contents of components are: potassium oxide per 0.1-1 g/100gFe; strontium oxide 0.1-1 g/100gFe; manganese oxide 1-20 g/100gFe, and rare earth metal oxides 1-10 g/100gFe; the rest is iron oxides. The molar ratio of ferric iron to double ferrous iron in the iron oxides, namely Fe³⁺/2Fe²⁺, is 0.4-1.5. The application aims to provide a molten iron catalyst with high strength, high activity, and high selectivity of higher α-olefin.

### FIELD OF THE DISCLOSURE

The present application relates to the technical field of chemical production, and particularly relates to a molten iron catalyst for high-temperature Fischer-Tropsch synthesis and a preparation method thereof, and its application in producing high-carbon α-olefin from synthesis gas.

### BACKGROUND

High-temperature Fischer-Tropsch synthesis products have high olefin content, especially the content of high value-added α-olefins, which are raw materials of fine chemicals that are in short supply in China. They can be used to synthesize fine chemicals like higher alcohols etc., providing more added value and diversity of Fischer-Tropsch synthesis products, as well as the anti-risk capability of the coal-to-liquids industry. However, the traditional catalyst systems cannot achieve high CO conversion and high olefin selectivity at the same time. Furthermore, there are additional issues relating to many by-products, such as methane, and high CO₂ selectivity. Therefore, it is necessary to improve the selectivity of high value-added α-olefins by adjusting the mechanism of action between active metals and cocatalysts.

### SUMMARY OF THE INVENTION

In view of the above technical problems, one of the objects of the present application is to provide a molten iron catalyst with high strength, high activity and high selectivity of higher α-olefin, and the technical solution provided is as follows:

A molten iron catalyst for producing high-carbon α-olefins from synthesis gas, comprising iron oxides and cocatalysts, characterized in that, mass contents of components are as follows:
potassium oxide 0.1-1 g/100 gFe; strontium oxide 0.1-1 g/100 gFe; manganese oxide 1-20 g/100 gFe and rare earth metal oxides 1-10 g/100 gFe; wherein the rest mass content is iron oxides; and, wherein, a molar ratio between ferric iron and double divalent iron, Fe³⁺/2Fe²⁺, in the iron oxides is 0.4-1.5.

In some embodiments, the iron oxides comprise a mixture phase of magnetite Fe₃O₄ and wustite FeO, and the molar ratio between ferric iron and double divalent iron, Fe³⁺/2Fe²⁺, in the iron oxides is 0.5-1.2.

In some embodiments, the mass contents of the components in the molten iron catalyst are as follows:
potassium oxide 0.25-0.8 g/100 gFe; strontium oxide 0.25-0.8 g/100 gFe; manganese oxide 2-15 g/100 gFe and rare earth metal oxides 2-6 g/100 gFe; wherein the rest mass content is iron oxides; and, the rare earth metal oxides are one or more from: cerium oxide, lanthanum oxide, samarium oxide and neodymium oxide.

The second object of the present application is to provide a preparation method of the above-mentioned molten iron catalyst which is unsophisticated in production technology, low in production cost and suitable for large-scale production, and the technical solution provided is as follows:

A preparation method for anyone of the above-mentioned molten iron catalysts, wherein the preparation method is a melting method, and comprises steps:

firstly, mixing the cocatalysts potassium carbonate, strontium carbonate, manganese carbonate and rare earth metal carbonate uniformly according to a mass ratio; and then, mixing the cocatalysts with a magnetite according to a mass ratio, before being loaded into a melting furnace; and undergoing successive processes of melting, cooling, crushing, ball milling and fractionating.

In some embodiments, the specific steps of the successive processes of melting, cooling, crushing, ball milling and fractionating comprise:
electric melting, with a melting time of 3-6 hr, under conditions including a melting voltage of 50-80V, a melting current of 1000-8000 A, a melting temperature of 1500-2000° C.;
wherein, after the melting is completed, the liquid melt is cooled immediately, and the solidified material is broken into pieces of 200-300 mm, and then the molten iron catalyst is obtained after jaw crushing, ball milling and multi-stage fractionating; wherein a particle size distribution of the molten iron catalyst is in the range of 10 to 250 microns with an average particle size of 40-70 microns.

The third object of the present application is to provide an application of the above-mentioned molten iron catalyst in the production of higher α-olefins from synthesis gas, which is suitable for Fischer-Tropsch synthesis to prepare higher α-olefins in fixed bed reactors and fluidized bed reactors. The reduction conditions of the molten iron catalyst are: reduction temperature at 300-400° C., reduction pressure at 1.0-3.0 MPa, H₂ as reduction gas, GHSV=4000-15000 hr⁻¹, and reduction time at 12-24 hr.

In some embodiments, the reaction conditions of the Fischer-Tropsch synthesis are: a reaction temperature of 280-400° C., and a reaction pressure of 1.0-3.0 MPa, synthesis gas ratio H₂/CO=0.6-3.0, and GHSV=1500-15000 hr⁻¹.

In some embodiments, a CO conversion rate of the Fischer-Tropsch synthesis is 80-98% per pass, and a selectivity of CH₄ is less than 10% and an α-olefin selectivity of C4+ is more than 40%.

The above technical solutions provided by the present application have at least the following beneficial effects:

1. A molten iron catalyst containing potassium oxide, strontium oxide, manganese oxide and rare earth metal oxides is prepared, wherein, the electron density on the surface of the active component, i.e. the iron oxide, is changed by the potassium oxide and the strontium oxide, thereby promoting the dissociative adsorption of CO, improving the conversion activity of CO. And alkali metal additives can also weaken the adsorption of H₂, thereby inhibiting the formation of methane and facilitating the growth of carbon chains. The manganese oxide can improve the reducibility of molten iron catalysts and the regeneration performance in synthesis gas, creating more active sites for CO dissociative adsorption on the iron-based surface. These active sites have strong carbonization ability, and can inhibit the hydrogenation reaction and increase the proportion of olefins in the product. In addition, by adding a small amount of rare earth metal oxides, the selectivity of heavy hydrocarbons in the product and the chain growth probability of the product can be increased. Due to the synergistic effect of the above various additives, the dissociative adsorption of H₂ and CO on the catalyst surface are facilitated, increasing the catalytic reaction activity, and facilitating the generation of olefins at the same time, thereby increasing the selectivity of higher α-olefins.

2. The raw materials for preparing the catalyst of the present application are cheap and easy to obtain, and the preparation process is simple, and the utilization rate of iron is high, which is suitable for industrial production.

3. The catalyst prepared by the melting method in the present application has high mechanical strength, outstanding wear resistance and impact resistance, and is especially suitable for fluidized bed reactors and fixed bed reactors.

4. The molten iron catalyst of the present application can achieve an efficient and direct conversion of synthesis gas to produce high-carbon α-olefins, and achieve a high-value utilization of synthesis gas conversion. The single-pass CO conversion rate of the catalyst is 80-98%, and the CH₄ selectivity is less than 10%, and the selectivity of C4+α-olefins is over 40%.

### DESCRIPTION OF THE ILLUSTRATED EMBODIMENTS

The present application will be further described below in conjunction with specific embodiments. It should be understood, that these examples are only used to illustrate the present application and not to limit the scope of the present application. In addition, it should be understood, that after reading the content taught by the present application, those skilled in the art can make various changes or modifications to the present application, and these equivalent solutions also fall within the scope defined by the appended claims of the present application. Various common reagents used in the examples are all commercially available products.

The molten iron catalyst provided in the present application can be prepared by a melting method and has high wear resistance, high impact resistance and crush resistance, and is especially suitable for Fischer-Tropsch synthesis processes in fluidized bed reactors or fixed bed reactors.

### Embodiment 1

Preparation steps: firstly, the additives including potassium carbonate 1.1 kg, strontium carbonate 1.2 kg, manganese carbonate 100 kg, hydrated cerium carbonate 12.5 kg and magnetite powder 600 kg, are mixed uniformly in a mixer, and the mixed powder is loaded into a melting furnace, wherein three electrodes are connected by iron bars, and energized for the melting process. The melting process is controlled by adjusting a melting voltage to maintain a melting current at about 7000 A, and the melting time is 3.5 hours; after the melting process is completed, the liquid melt is introduced into a cooling tank, and cooled immediately to the room temperature, which is then initially broken into 200-300 mm blocks, and a catalyst A is obtained finally through the process of jaw crushing, ball milling and two-stage fractionating. The particle size distribution is in the range of 10 to 250 microns, and the average particle size is 45 microns. The composition of catalyst A is: Fe³⁺/2Fe²⁺=0.55, potassium oxide 0.17 g/100 gFe, strontium oxide 0.20 g/100 gFe, manganese oxide 17.8 g/100 gFe, cerium oxide 1.1 g/100 gFe; and the mass content of Fe is 70.2%.

The catalytic process of Fischer-Tropsch synthesis: Catalyst A undergoes the reduction process at first, and the reduction conditions are: 400° C., 2.0 MPa, and gaseous hourly space velocity (GHSV) of 5000 hr⁻¹. The reducing material is pure H₂. After a reduction time of 12 hours, the synthesis reaction is carried out. The synthesis conditions are: 340° C., 2.0 MPa, H₂/CO ratio of 3.0, and space velocity of 5000 hr⁻¹.

Through using the molten iron catalyst of this embodiment, the Fischer-Tropsch synthesis process has a CO conversion rate of 80.5%, a methane selectivity of 7.8 wt %, a C2-C3 hydrocarbon selectivity of 12.4 wt %, and a C4+α-olefin selectivity of 46.5 wt %.

### Embodiment 2

Preparation steps: firstly, the additives including potassium carbonate 2.5 kg, strontium carbonate 2.45 kg, manganese carbonate 56.4 kg, hydrated cerium carbonate 114 kg and magnetite powder 600 kg are mixed uniformly in a mixer, and the mixed powder is loaded into a melting furnace, wherein three electrodes are connected by iron bars, and energized for the melting process. The melting process is controlled by adjusting a melting voltage to maintain a melting current at about 7000 A, and the melting time is 5 hours; after the melting process is completed, the liquid melt is introduced into a cooling tank, and cooled immediately to the room temperature, which is then initially broken into 200-300 mm blocks, and a molten iron catalyst B is obtained finally through the process of jaw crushing, ball milling, and two-stage fractionating. The molten iron catalyst B has a particle size distribution of 10-250 microns and an average particle size of 55 microns. The composition of catalyst B is: Fe³⁺/2Fe²⁺=0.45, potassium oxide 0.40 g/100 gFe, strontium oxide 0.40 g/100 gFe, manganese oxide 10.0 g/100 gFe, cerium oxide 10.0 g/100 gFe, and the mass content of Fe is 69.7%.

The catalytic process of Fischer-Tropsch synthesis: Catalyst B undergoes the reduction process at first. The reduction conditions are: 300° C., 3.0 MPa, space velocity of 10000 hr⁻¹. The reducing material is pure H₂. After a reduction time of 24 hours, the synthesis reaction is carried out. The synthesis conditions are: 330° C., 2.4 MPa, H₂/CO ratio of 3.0, and space velocity of 2500 hr⁻¹.

Through using the molten iron catalyst of this embodiment, the Fischer-Tropsch synthesis process has a CO conversion rate of 98.5%, a methane selectivity of 5.9 wt %, a C2-C3 hydrocarbon selectivity of 17.4 wt %, and a C4+α-olefin selectivity of 52.5 wt %.

### Embodiment 3

Preparation steps: firstly, the additives including potassium carbonate 6.0 kg, strontium carbonate 3.2 kg, manganese carbonate 110 kg, hydrated cerium carbonate 35 kg and magnetite powder 600 kg are mixed uniformly in a mixer, and the mixed powder is loaded into a melting furnace, wherein three electrodes are connected by iron bars, and energized for the melting process. The melting process is controlled by adjusting a melting voltage to maintain a melting current at about 7000 A, and the melting time is 3 hours; and after the melting process is completed, the liquid melt is introduced into a cooling tank, and cooled immediately to the room temperature, which is then initially broken into 200-300 mm blocks, and a molten iron catalyst C is obtained finally through the process of jaw crushing, ball milling, and two-stage fractionating. The molten iron catalyst C has a particle size distribution of 10-250 microns and an average particle size of 68 microns. The composition of catalyst C is: Fe³⁺/2Fe²⁺=1.15, potassium oxide 0.95 g/100 gFe, strontium oxide 0.53 g/100 gFe, manganese oxide 19.5 g/100 gFe, cerium oxide 3.0 g/100 gFe, and the mass content of Fe is 69.1%.

The catalytic process of Fischer-Tropsch synthesis: Catalyst C undergoes the reduction process at first. The reduction conditions are: 370° C., 1.5 MPa, space velocity of 15000 hr⁻¹. The reducing material is pure H₂. After a reduction time of 24 hours, the synthesis reaction is carried out. The synthesis conditions are: 350° C., 3.0 MPa, H₂/CO ratio of 2.0, and space velocity of 5000 hr⁻¹.

Through using the molten iron catalyst of this embodiment, the Fischer-Tropsch synthesis process has a CO conversion rate of 83.1%, a methane selectivity of 9.85 wt %, a C2-C3 hydrocarbon selectivity of 23.5 wt %, and a C4+α-olefin selectivity of 56.8 wt %.

### Embodiment 4

Preparation steps: firstly, the additives including potassium carbonate 3.5 kg, strontium carbonate 6.0 kg, manganese carbonate 28.5 kg, hydrated cerium carbonate 57 kg and magnetite powder 600 kg are mixed uniformly in a mixer, and the mixed powder is loaded into a melting furnace, wherein three electrodes are connected by iron bars, and energized for the melting process. The melting process is controlled by adjusting a melting voltage to maintain a melting current at about 7000 A, and the melting time is 6 hours; after the melting process is completed, the liquid melt is introduced into a cooling tank, and cooled immediately to the room temperature, which is then initially broken into 200-300 mm blocks, and a molten iron catalyst D is obtained finally through the process of jaw crushing, ball milling, and two-stage fractionating. The molten iron catalyst D has a particle size distribution of 10-250 microns and an average particle size of 52 microns. The composition of catalyst D is: Fe³⁺/2Fe²⁺=1.0, potassium oxide 0.56 g/100 gFe, strontium oxide 0.99 g/100 gFe, manganese oxide 5.0 g/100 gFe, cerium oxide 5.0 g/100 gFe, and the mass content of Fe is 69.8%.

The catalytic process of Fischer-Tropsch synthesis: Catalyst D undergoes the reduction process at first. The reduction conditions are: 340° C., 2.1 MPa, space velocity of 5000 hr⁻. The reducing material is pure H₂. After a reduction time of 18 hours, the synthesis reaction is carried out. The synthesis conditions are: 340° C., 2.1 MPa, H₂/CO ratio of 3.6, and space velocity of 4500 hr⁻¹.

Through using the molten iron catalyst of this embodiment, the Fischer-Tropsch synthesis process has a CO conversion rate of 93.1%, a methane selectivity of 5.65 wt %, a C2-C3 hydrocarbon selectivity of 17.1 wt %, and a C4+α-olefin selectivity of 59.1 wt %.

### Embodiment 5

Preparation steps: firstly, the additives including potassium carbonate 1.5 kg, strontium carbonate 3.0 kg, manganese carbonate 20 kg, hydrated lanthanum carbonate 12 kg and magnetite powder 600 kg are mixed uniformly in a mixer, and the mixed powder is loaded into a melting furnace, wherein three electrodes are connected by iron bars, and energized for the melting process. The melting process is controlled by adjusting a melting voltage to maintain a melting current at about 6000 A, and the melting time is 5.5 hours; after the melting process is completed, the liquid melt is introduced into a cooling tank, and cooled immediately to the room temperature, which is then initially broken into 200-300 mm blocks, and a molten iron catalyst E is obtained finally through the process of jaw crushing, ball milling, and two-stage fractionating. The molten iron catalyst E has a particle size distribution of 10-250 microns and an average particle size of 48 microns. The composition of catalyst E is: Fe³⁺/2Fe²⁺=0.9, potassium oxide 0.24 g/100 gFe, strontium oxide 0.49 g/100 gFe, manganese oxide 3.6 g/100 gFe, lanthanum oxide 2.0 g/100 gFe, and the mass content of Fe is 70.8%.

The catalytic process of Fischer-Tropsch synthesis: Catalyst E undergoes the reduction process at first. The reduction conditions are: 320° C., 3.0 MPa, space velocity of 8000 hr⁻. The reducing material is pure H₂. After a reduction time of 22 hours, the synthesis reaction is carried out. The synthesis conditions are: 330° C., 1.0 MPa, H₂/CO ratio of 2.0, and space velocity of 3000 hr⁻¹.

Through using the molten iron catalyst of this embodiment, the Fischer-Tropsch synthesis process has a CO conversion rate of 95.2%, a methane selectivity of 5.65 wt %, a C2-C3 hydrocarbon selectivity of 18.5 wt %, and a C4+α-olefin selectivity of 52.8 wt %.

### Embodiment 6

Preparation steps: firstly, the additives including potassium carbonate 2.5 kg, strontium carbonate 1.0 kg, manganese carbonate 80 kg, hydrated lanthanum carbonate 36 kg and magnetite powder 600 kg are mixed uniformly in a mixer, and the mixed powder is loaded into a melting furnace, wherein three electrodes are connected by iron bars, and energized for the melting process. The melting process is controlled by adjusting a melting voltage to maintain a melting current at about 6500 A, and the melting time is 4.5 hours; after the melting process is completed, the liquid melt is introduced into a cooling tank, and cooled immediately to the room temperature, which is then initially broken into 200-300 mm blocks, and a molten iron catalyst F is obtained finally through the process of jaw crushing, ball milling, and two-stage fractionating. The molten iron catalyst F has a particle size distribution of 10-250 microns and an average particle size of 52 microns. The composition of catalyst F is: Fe³⁺/2Fe²⁺=0.82, potassium oxide 0.4 g/100 gFe, strontium oxide 0.16 g/100 gFe, manganese oxide 14.2 g/100 gFe, lanthanum oxide 6.0 g/100 gFe, and the mass content of Fe is 69.3%.

The catalytic process of Fischer-Tropsch synthesis: Catalyst F undergoes the reduction process at first. The reduction conditions are: 370° C., 0.5 MPa, space velocity of 5000 hr⁻. The reducing material is pure H₂. After a reduction time of 15 hours, the synthesis reaction is carried out. The synthesis conditions are: 340° C., 1.5 MPa, H₂/CO ratio of 1.6, and space velocity of 5000 hr⁻¹.

Through using the molten iron catalyst of this embodiment, the Fischer-Tropsch synthesis process has a CO conversion rate of 89.2%, a methane selectivity of 4.65 wt %, a C2-C3 hydrocarbon selectivity of 16.4 wt %, and a C4+α-olefin selectivity of 56.8 wt %.

It can be known from the above embodiments:

1. The catalyst in this application is used in the Fischer-Tropsch synthesis process. Through the structure and composition of its active components, through the type and ratio of cocatalysts, and through the comprehensive design of the specific preparation methods and technology of the catalyst, multiple groups of implementations are provided to obtain the catalyst with high strength, high activity, and high selectivity of higher α-olefin.

2. The basicity of the catalyst surface is improved by alkali metal oxides (potassium oxide and strontium oxide), which facilitates the growth of carbon chains. The hydrogenation reaction on the catalyst surface is inhibited by manganese oxide as a structural aid, and the proportion of olefin products is increased. Through the addition of a small amount of rare metal oxides, the selectivity of heavy hydrocarbons is increased, and the chain growth probability of the product is increased. Due to the synergistic effect of the above various additives, the dissociative adsorption of H₂ and CO on the catalyst surface is facilitated, increasing the catalytic reaction activity, and facilitating the formation of olefins simultaneously, and improving the selectivity of higher α-olefins.

3. Using the mixture of magnetite Fe₃O₄ and wustite FeO as the active components of the catalyst in the reaction process of producing low-carbon olefins from synthesis gas, catalytic reaction activities are improved, and the high-value utilization of synthesis gas conversion is achieved. The conversion rate is 80-98%, and the selectivity of CH₄ is less than 10%, and the selectivity of α-olefins above C4 is more than 40%.

The above-mentioned embodiments only represent several embodiments of the present application, and the descriptions thereof are specific and detailed, but should not be construed as a limitation on the scope of the patent of the present application. It should be pointed out, that for those of ordinary skill in the art, without departing from the concept of the present application, several modifications and improvements can also be made, which all belong to the protection scope of the present application. Therefore, the scope of protection of the present application should be determined by the appended claims.

1. A molten iron catalyst for producing high-carbon α-olefins from synthesis gas, comprising iron oxides and cocatalysts, characterized in that, mass contents of components are as follows:
potassium oxide 0.1-1 g/100 gFe; strontium oxide 0.1-1 g/100 gFe; manganese oxide 1-20 g/100 gFe and rare earth metal oxides 1-10 g/100 gFe; wherein the rest mass content is iron oxides.

2. The molten iron catalyst according to claim 1, wherein the molar ratio between ferric iron and double divalent iron Fe³⁺/2Fe²⁺ in the iron oxides is 0.5-1.2.

3. The molten iron catalyst according to claim 1, wherein the mass contents of the components in the molten iron catalyst are as follows:
potassium oxide 0.25-0.8 g/100 gFe; strontium oxide 0.25-0.8 g/100 gFe; manganese oxide 2-15 g/100 gFe and rare earth metal oxides 2-6 g/100 gFe; wherein the rest mass content is iron oxides.

4. A preparation method for a molten iron catalyst, wherein the preparation method is a melting method, and comprises steps:
firstly, mixing cocatalysts potassium carbonate, strontium carbonate, manganese carbonate and rare earth metal carbonate uniformly according to a mass ratio; and then, mixing the cocatalysts with a magnetite according to a mass ratio, before being loaded into a melting furnace; and undergoing successive processes of melting, cooling, crushing, ball milling and fractionating.

5. The preparation method according to claim 4, wherein specific steps of the successive processes of melting, cooling, crushing, ball milling and fractionating comprise:
electric melting, with a melting time of 3-6 hr, under conditions including a melting voltage of 50-80V, a melting current of 1000-8000 A, a melting temperature of 1500-2000° C.;
wherein, after the melting is completed, the liquid melt is cooled immediately, and the solidified material is broken into pieces of 200-300 mm, and then the molten iron catalyst is obtained after jaw crushing, ball milling and multi-stage fractionating; wherein a particle size distribution of the molten iron catalyst is in the range of 10 to 250 microns with an average particle size of 40-70 microns.

6. An application of a molten iron catalyst, characterized in that,
mass contents of components of the molten iron catalyst are as follows:
potassium oxide 0.1-1 g/100 gFe; strontium oxide 0.1-1 g/100 gFe; manganese oxide 1-20 g/100 gFe and rare earth metal oxides 1-10 g/100 gFe; wherein the rest mass content is iron oxides; and,
the molten iron catalyst is applied in producing high-carbon α-olefins by Fischer-Tropsch synthesis in a fixed-bed reactor or in a fluidized-bed reactor.

7. The application of a molten iron catalyst according to claim 6, wherein reduction conditions of the molten iron catalyst are: a reduction temperature of 300-400° C., a reduction pressure of 1.0-3.0 MPa, a reduction material H₂, GHSV=4000-15000 hr⁻¹ and a reduction time of 12-24 hr.

8. The application of a molten iron catalyst in the production according to claim 6, wherein reaction conditions of the Fischer-Tropsch synthesis are: a reaction temperature of 280-400° C., and a reaction pressure of 1.0-3.0 MPa, synthesis gas ratio H₂/CO=0.6-3.0, and GHSV=1500-15000 hr⁻¹.

9. The application of a molten iron catalyst according to claim 6, wherein a CO conversion rate of the Fischer-Tropsch synthesis is 80-98% per pass, and a selectivity of CH₄ is less than 10% and an α-olefin selectivity of C4+ is more than 40%.

10. The molten iron catalyst according to claim 1,
wherein, a molar ratio between ferric iron and double divalent iron Fe³⁺/2Fe²⁺ in the iron oxides is 0.4-1.5.

11. The molten iron catalyst according to claim 1,
wherein, the iron oxides comprise a mixture phase of magnetite Fe₃O₄ and wustite FeO.

12. The molten iron catalyst according to claim 1,
wherein the rare earth metal oxides are one or more from: cerium oxide, lanthanum oxide, samarium oxide and neodymium oxide.

13. The preparation method according to claim 4,
wherein the iron oxides comprise a mixture phase of magnetite Fe₃O₄ and wustite FeO, and the molar ratio between ferric iron and double divalent iron Fe³⁺/2Fe²⁺ in the iron oxides is 0.4-1.5.

14. The preparation method according to claim 4,
wherein the iron oxides comprise a mixture phase of magnetite Fe₃O₄ and wustite FeO, and the molar ratio between ferric iron and double divalent iron Fe³⁺/2Fe²⁺ in the iron oxides is 0.5-1.2.

15. The preparation method according to claim 4,
wherein mass contents of components of the molten iron catalyst are as follows:
potassium oxide 0.1-1 g/100 gFe; strontium oxide 0.1-1 g/100 gFe; manganese oxide 1-20 g/100 gFe and rare earth metal oxides 1-10 g/100 gFe; wherein the rest mass content is iron oxides.

16. The application of a molten iron catalyst according to claim 6,
wherein the iron oxides comprise a mixture phase of magnetite Fe₃O₄ and wustite FeO, and the molar ratio between ferric iron and double divalent iron Fe³⁺/2Fe²⁺ in the iron oxides is 0.4-1.5.

17. The application of a molten iron catalyst according to claim 6,
wherein the iron oxides comprise a mixture phase of magnetite Fe₃O₄ and wustite FeO, and the molar ratio between ferric iron and double divalent iron Fe³⁺/2Fe²⁺ in the iron oxides is 0.5-1.2.
